(19) United States
(12) Patent Application Publication
(10) Pub. No.: US 2017/0000428 A1
(43) Pub. Date: Jan. 5, 2017

(54) X-RAY CT APPARATUS AND CONTRAST IMAGING METHOD

(71) Applicant: HITACHI, LTD., Tokyo (JP)

(72) Inventor: Taiga GOTO, Tokyo (JP)

(73) Assignee: HITACHI, LTD., Tokyo (JP)

(21) Appl. No.: 15/102,597

(22) PCT Filed: Jan. 8, 2015
(86) PCT No.: PCT/JP2015/050304

§ 371 Date: Jun. 8, 2016

(30) Foreign Application Priority Data
Jan. 15, 2014 (JP) 2014-005202

(51) Int. Cl. A61B 6/02; A61B 6/00; A61B 6/03

(52) U.S. Cl. CPC A61B 6/027; A61B 6/035; A61B 6/541

(57) ABSTRACT

In order to provide an X-ray CT apparatus and a contrast enhanced scanning method that can obtain an image having a favorable contrast effect without re-injecting contrast medium even when a scanning position overtakes a position of the contrast medium during scanning using the contrast medium, the X-ray CT apparatus determines whether or not a current scanning position overtakes the contrast medium during main scanning, resets a scanning condition when overtaking the contrast medium, executes re-scanning under the reset scanning condition, and determines overtaking of the contrast medium based on differential data between measurement data acquired by non-contrast enhanced scanning to be performed before the main scanning and measurement data to be acquired by the main scanning. Hence, no image needs to be reconstructed during scanning to check whether or not there is a contrast effect, and the overtaking determination is performed at a high speed, which can swiftly shift to re-scanning.

### TECHNICAL FIELD

The present invention relates to an X-ray CT apparatus and a contrast enhanced scanning method and, in detail, to the X-ray CT apparatus that can perform scanning using contrast medium.

### BACKGROUND ART

Conventionally, in an examination using an X-ray CT (Computed Tomography) apparatus, contrast CT examinations that perform scanning while contrast medium is being injected into an object are performed in order to obtain images with shades appropriate for diagnosis. In the contrast CT examinations, the injected contrast medium is delivered to the entire body through the blood flow, and when the contrast medium arrives at a scanning site, scanning using the X-ray CT apparatus starts. Whether or not the contrast medium has arrived at the scanning site is determined by, for example, a monitoring scan that monitors a density change of the contrast medium in a predetermined region of interest. Patent Literature 1 discloses a technique for measuring a density (CT value) of contrast medium in a region of interest from an image obtained by the monitoring scan to automatically switch to contrast enhanced scanning that is main scanning by determining that the contrast medium has arrived at a scanning site when the density exceeds a predetermined threshold value.

By the way, recent X-ray CT apparatuses can perform high-speed scanning. For example, when a size of an X-ray detector in the body-axis direction is approximately 20 to 40 [mm], a scanning condition such as the scanner rotation speed: 0.5 [s/rotation] and the helical pitch: approximately 0.8 to 1.3 are used. Under such a condition, the bed moving speed is 32 to 150 [mm/s], which can perform scanning at a higher speed than the average blood flow speed. Therefore, when a flow of contrast medium is slower than the estimate flow, there is a case where a scanning position overtakes the contrast medium. Continuing scanning in the state where the scanning position has overtaken the contrast medium cannot obtain a desired contrast effect, which can result in that examination needs to be performed again. This is a heavy burden for a patient, and it is undesirable. Therefore, Patent Literature 1 describes that the bed moving speed and the scanner rotation speed are controlled based on a CT value of a blood vessel portion in a CT image reconstructed during contrast enhanced scanning.

### CITATION LIST

### Patent Literature

PTL 1: Japanese Unexamined Patent Publication No. 2005-160784

### SUMMARY OF INVENTION

### Technical Problem

However, the method of PTL 1 reconstructs an image during contrast enhanced scanning and measures a CT value in the image to determine a threshold value. Therefore, it takes a long time to perform a calculation process for the image reconstruction, the timing to obtain overtaking results of contrast medium is delayed, and then a scanning position advances in the meantime, which results in that a contrast failure portion is generated. Even in case of overtaking the contrast medium, it is desired that a satisfactory image can be obtained by one examination without injecting the contrast medium again.

The present invention was made in consideration with the above problems, and the purpose is to provide an X-ray CT apparatus and a contrast enhanced scanning method that can obtain an image having a satisfactory contrast effect without re-injecting contrast medium even in a case where a scanning position overtakes a position of the contrast medium during scanning using the contrast medium.

### Solution to Problem

In order to achieve the above purpose, the first invention is an X-ray CT apparatus characterized by comprising: an X-ray source that irradiates X-rays to an object; an X-ray detector that is disposed opposite to the X-ray source and detects the X-rays transmitted through the object; a rotary disk that is provided with the X-ray source and the X-ray detector and rotates around the object; an image reconstruction unit that reconstructs an image based on the transmitted X-ray data detected by the X-ray detector; a scanning condition setting unit that sets a scanning condition of main scanning to be performed by injecting contrast medium into the object; a main scanning control unit that executes the main scanning under the scanning condition set by the scanning condition selling unit; an overtaking determination unit that determines whether or not a scanning position overtakes a position of the contrast medium during executing the main scanning; and a re-scanning control unit that resets the scanning condition and executes re-scanning under the reset scanning condition in a case where overtaking is determined by the overtaking determination unit.

Also, the second invention is a contrast enhanced scanning method characterized by comprising steps of: executing main scanning to be performed by injecting contrast medium into an object under set a scanning condition by an X-ray CT apparatus; determining overtaking of a scanning position for a position of contrast medium during the main scanning; setting the scanning condition again in case of determining overtaking; and executing re-scanning under the reset scanning condition.

### Advantageous Effects of Invention

The present invention can provide an X-ray CT apparatus and a contrast enhanced scanning method that can obtain an image having a satisfactory contrast effect without re-injecting contrast medium even in a case where a scanning position overtakes a position of the contrast medium during scanning using the contrast medium.

### BRIEF DESCRIPTION OF DRAWINGS

FIG. 1 is an overall configuration diagram of an X-ray CT apparatus 1.

FIG. 2 is a diagram explaining scanning trajectories.

FIG. 3 is a flow chart showing a procedure for contrast scanning processing executed by the X-ray CT apparatus 1 of the present invention.

FIG. 4 is an example of a condition setting window 3.

FIG. 5 is a configuration diagram of an overtaking determination unit 128.

FIG. 6(a) shows a position where it was determined that contrast medium had been overtaken (contrast overtaking determining position) and an example of a scanning range, FIG. 6(b) shows a bed moving direction of turnaround scanning, and FIG. 6(c) shows a bed moving direction of forward-direction scanning.

FIG. 7 is a diagram explaining scanning trajectories.

FIG. 8 is a diagram showing CT value transitions when using contrast medium (Time Density Curve).

### DESCRIPTION OF EMBODIMENTS

Hereinafter, an embodiment of the present invention will be described in detail by referring to the drawings.

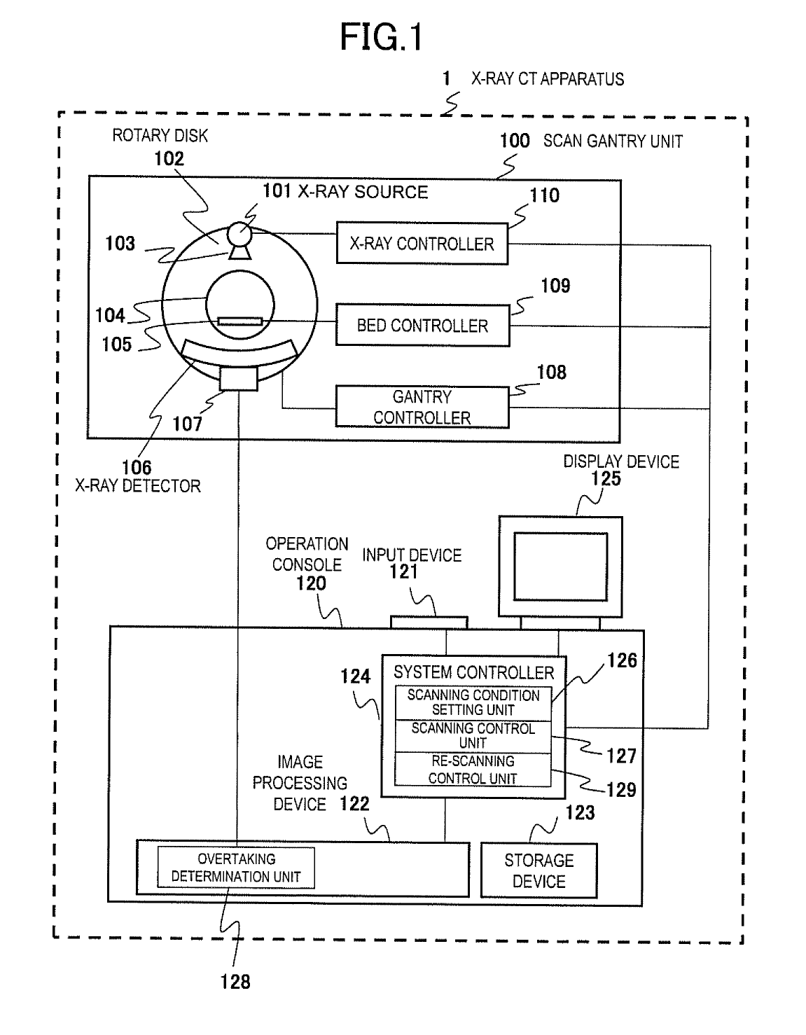

First, referring to FIG. 1, the overall configuration of the X-ray CT apparatus 1 will be described.

As shown in FIG. 1, the X-ray CT apparatus 1 comprises a scan gantry unit 100, a bed 105, and an operation console 120. The scan gantry unit 100 is a device that irradiates X-rays to an object and detects the X-rays transmitted through the object. The operation console 120 is a device that controls each part of the scan gantry unit 100 and acquires the transmitted X-ray data measured by the scan gantry unit 100 to generate an image. The bed 105 is a device that places the object and carries the object in and out of an X-ray irradiation range of the scan gantry unit 100.

The scan gantry unit 100 comprises an X-ray source 101, a rotary disk 102, a collimator 103, an X-ray detector 106, a data acquisition system 107, a gantry controller 108, a bed controller 109, and an X-ray controller 110.

The operation console 120 comprises an input device 121, an image processing device 122, a storage device 123, a system controller 124, and a display device 125.

The rotary disk 102 of the scan gantry unit 100 is provided with an opening 104, and the X-ray source 101 and the X-ray detector 106 are disposed opposite to each other across the opening 104. An object placed on the bed 105 is inserted into the opening 104. The rotary disk 102 rotates around the object by a driving force transmitted from a rotary disk driving device through a driving transmission system. The rotary disk driving device is controlled by the gantry controller 108.

The X-ray source 101 is controlled by the X-ray controller 110 and irradiates X-rays continuously or intermittently at a predetermined intensity. The X-ray controller 110 controls an X-ray tube voltage to be applied to and an X-ray tube current to be supplied to the X-ray source 101 according to the X-ray tube voltage and the X-ray tube current determined by the system controller 124 of the operation console 120.

The collimator 103 is provided in an X-ray irradiation opening of the X-ray source 101. The collimator 103 restricts an irradiation range of an X-ray radiated from the X-ray source 101. For example, the X-ray is shaped into a corn beam and the like (a cone or pyramid shape). The opening width of the collimator 103 is controlled by the system controller 124.

X-rays are irradiated from the X-ray source 101, pass through the collimator 103, and transmit through an object before entering the X-ray detector 106.

In the X-ray detector 106, for example, a group of X-ray detection elements is composed by combining scintillators and photodiodes. Approximately 1,000 pieces of the elements are arranged in the channel direction (circumference direction), and approximately 1 to 320 pieces of the elements are arranged in the column direction (body-axis direction). The X-ray detector 106 is disposed so as to be opposed to the X-ray source 101 across an object. The X-ray detector 106 detects an amount of X-rays irradiated from the X-ray source 101 and transmitted through the object in order to output to the data acquisition system 107.

The data acquisition system 107 collects an X-ray amount to be detected by the respective X-ray detection elements of the X-ray detector 106, converts the amount into digital data, and outputs it as transmission X-ray data to the image processing device 122 of the operation console 120 in order.

The image processing device 122 acquires the transmission X-ray data input from the data acquisition system 107 and performs pre-processing such as logarithmic transformation, sensitivity correction, and the like in order to generate projection data required for reconstruction. Also, the image processing device 122 uses the generated projection data to reconstruct object images such as tomographic images.

The system controller 124 stores the object images reconstructed by the image processing device 122 in the storage device 123 and displays it on the display device 125.

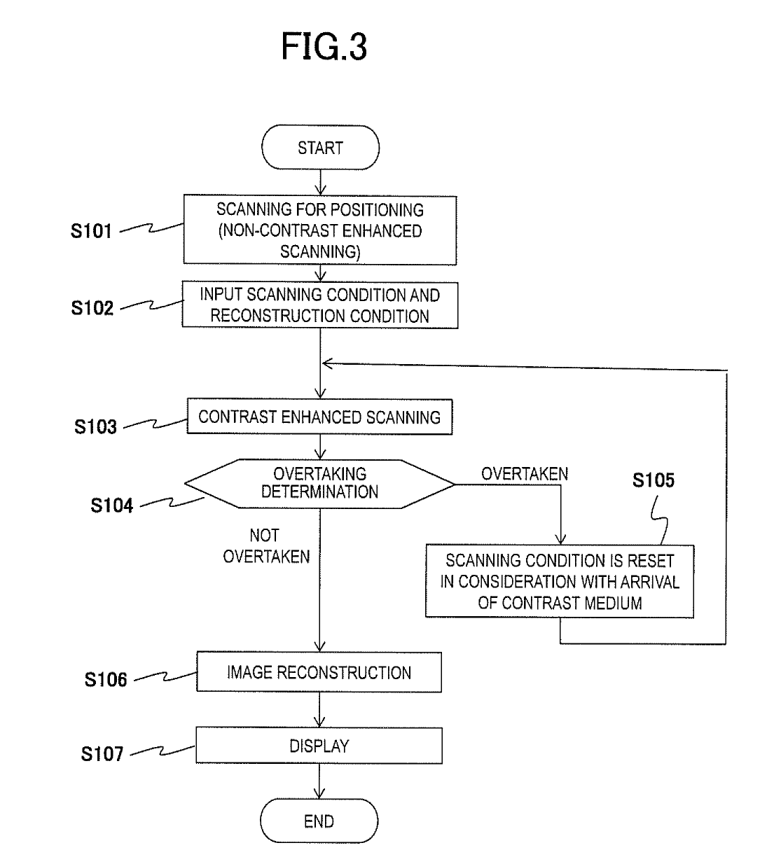

The system controller 124 is a computer comprising a CPU (Central Processing Unit), a ROM (Read Only Memory), a RAM (Random Access Memory), and the like. The storage device 123 is a data storage device such as a hard disk and stores programs, data, and the like to achieve functions of the X-ray CT apparatus 1 in advance. The system controller 124 performs contrast enhanced scanning processing according to the processing procedure shown in FIG. 3. The contrast enhanced scanning processing will be described in detail later.

The display device 125 is composed of a display such as a liquid crystal panel or a CRT monitor and a logical circuit to execute display processing in cooperation with the display and is connected to the system controller 124. The display device 125 displays object images to be output from the image processing device 122 and various information to be handled by the system controller 124.

The input device 121 is composed of, for example, pointing devices such as a keyboard and a mouse, a numeric keypad, and various switch buttons and outputs various commands and information to be input by an operator to the system controller 124. The operator operates the X-ray CT apparatus 1 interactively using the display device 125 and the input device 121. The input device 121 may be a touch panel-type input device that is integrally composed with a display screen of the display device 125.

The bed 105 comprises a top plate on which an object is placed, a vertical movement device, and a top plate driving device, moves the top plate up and down, moves back and forth in the body-axis direction, and moves left and right in a vertical direction to the body axis and a parallel direction (horizontal direction) to the floor surface by control of the bed controller 109. During scanning, the bed controller 109 moves the top plate at a bed moving speed and in a moving direction determined by the system controller 124.

Next, a functional configuration related to contrast enhanced scanning will be described.

The X-ray CT apparatus 1 of the present invention comprises a scanning condition setting unit 126, a scanning control unit 127, an overtaking determination unit 128, and a re-scanning control unit 129 as the functional configuration related to imaging using contrast medium (hereinafter, referred to as contrast enhanced scanning). It is desired to provide the system controller 124 with the scanning condition setting unit 126, the scanning control unit 127, and the re-scanning control unit 129. Although it is desired to provide the image processing device 122 with the overtaking determination unit 128 from the viewpoint of increasing a determination speed, the overtaking determination unit 128 may be provided with the system controller 124.

The scanning condition setting unit 126 sets a scanning condition and a reconstruction condition for positioning scanning and main scanning. The X-ray CT apparatus 1 of the present embodiment performs contrast enhanced scanning that performs scanning while contrast medium is being injected into an object in main scanning. For this purpose, the scanning condition setting unit 126 sets a scanning condition for contrast enhanced scanning as those for main scanning. The scanning condition includes an X-ray condition such as a scanning range, a region of interest, an X-ray tube voltage, and an X-ray tube current, a gantry rotation speed, a bed speed, a helical pitch, and the like. The reconstruction condition includes a reconstruction FOV, a reconstruction slice thickness, and the like. The scanning condition is input by an operator through the input device 121 of the operation console 120. Each of the input conditions is stored in the storage device 123. Also, in a case where a scanning position overtakes contrast medium during contrast enhanced scanning, the scanning condition setting unit 126 resets a scanning condition in consideration with arrival of the contrast medium.

The scanning control unit 127 controls each part of the scan gantry unit 100 and the bed 105 based on the scanning condition set by the scanning condition setting unit 126 and executes scanning. Specifically, the scanning control unit 127 transmits control signals to the X-ray controller 110, the gantry controller 108, and the bed controller 109 based on the scanning condition. The X-ray controller 110 controls an electric power to be input to the X-ray source 101 based on the control signals input from the system controller 124. The gantry controller 108 controls a driving system of the rotary disk 102 according to the scanning condition including a rotation speed to rotate the rotary disk 102. The bed controller 109 adjusts a position of the bed 105 to a predetermined scanning start position based on a set scanning range and moves the top plate of the bed 105 at a predetermined speed based on the scanning condition including a bed speed (helical pitch) during scanning.

When obtaining measurement data (raw data of transmission X-ray data) from the data acquisition system 107 during contrast enhanced scanning, the overtaking determination unit 128 determines whether or not a current scanning position overtakes a position of contrast medium in an object using the measurement data. In the present invention, the overtaking determination unit 128 performs difference processing for measurement data acquired by non-contrast enhanced scanning to be performed before main scanning and measurement data acquired by main scanning between data in which a body-axis direction position and a view position are the same, and then overtaking of the contrast medium is determined based on the differential data.

The non-contrast enhanced scanning is performed without injecting contrast medium. For example, measurement data of scanogram scanning for positioning can be used for overtaking determination.

Also, after the measurement data is acquired by corresponding trajectories of non-contrast enhanced scanning and contrast enhanced scanning (main scanning), the overtaking determination may be performed based on differential data between the measurement data of the non-contrast enhanced scanning and the contrast enhanced scanning.

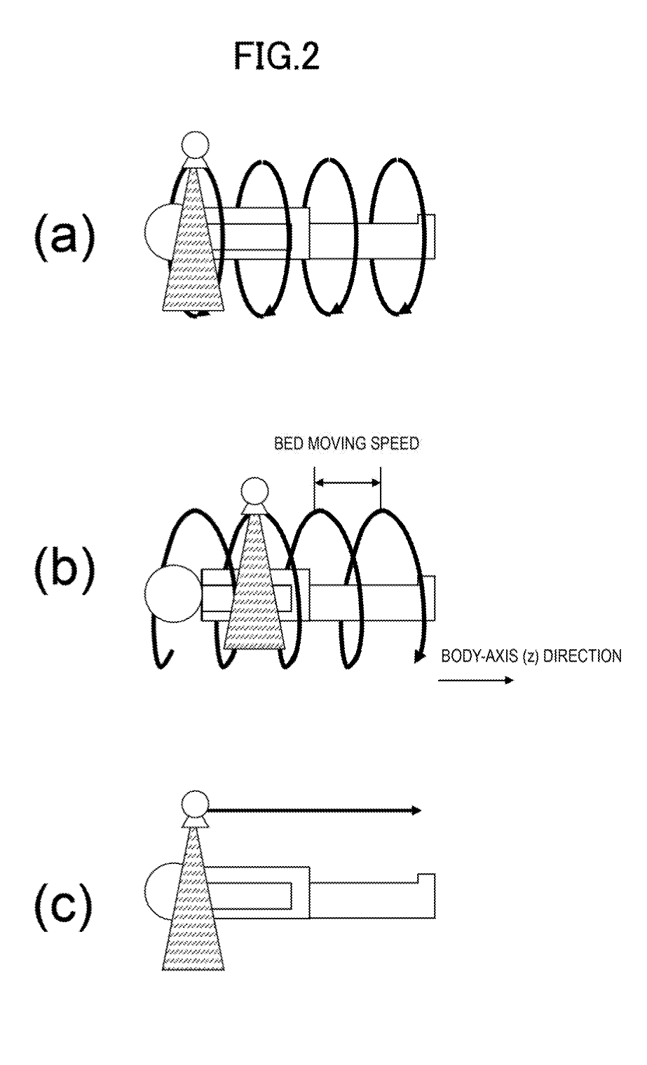

Referring to FIG. 2, the scanning trajectories will be described.

Scanning methods of the X-ray CT apparatus 1 include an axial scan (referred also to as a circle scan) shown in FIG. 2(a), a spiral scan (referred also to as a helical scan) shown in FIG. 2(b), scanogram (scanogram scanning) shown in FIG. 2(c), and the like.

In the axial scan shown in FIG. 2(a), a table position of the bed 105 is fixed, and the X-ray source 101 and the X-ray detector 106 that are disposed opposite to each other rotate around an object. In the helical scan shown in FIG. 2(b), the X-ray source 101 and the X-ray detector 106 rotate around the object while the table position of the bed 105 is being moved parallel in the body-axis direction. The helical scan is used for scanning a wide range in the body-axis direction.

In the scanogram scanning shown in FIG. 2(c), the X-ray source 101 and the X-ray detector 106 move parallel to the object in the body-axis direction in order to acquire measurement data. Generally, the scanogram scanning is performed before main scanning for positioning to determine a range for tomographic image generation (scanning range) and for a reference to calculate a modulation curve when a tube current is modulated to reduce an exposure dose.

In a case of using measurement data obtained by the scanogram scanning shown in FIG. 2(c) for overtaking determination, difference processing is performed between measurement data obtained by the scanogram scanning (hereinafter, referred to as scanogram measurement data) and measurement data obtained by main scanning (referred to as main scanning measurement data) in a view position of the scanogram scanning (X-ray tube position). Therefore, the frequency of the overtaking determination is once per rotation of the rotary disk.

On the other hand, in a case of performing the overtaking determination using, non-contrast enhanced scanning in which trajectories were synchronized, view positions (tube positions) are always the same in the same body-axis direction position, and all the measurement data is can be used for the overtaking determination. Therefore, the frequency of the overtaking determination can be improved, which can obtain results of the overtaking determination at an early timing.

The overtaking determination unit 128 performs difference processing between measurement data for each element in a predetermined range and performs integration processing for the differential data. Then, in a case where the integration value is larger than a predetermined threshold value, it is determined that overtaking has not occurred because there is a difference from measurement data of non-contrast enhanced scanning (the contrast effect is found). In a case where the integration value is equal to or less than a predetermined threshold value, it is determined that overtaking occurred because there is a little difference from measurement data of non-contrast enhanced scanning (the contrast effect is not found).

In a case of performing overtaking determination using measurement data (raw data), an image does not need to be reconstructed for the overtaking determination during scanning. This speeds up calculation processing, which can detect overtaking immediately. Difference processing and integration processing does not always need to be performed for all the elements but may be performed for a partial range such as the measurement center part or a region of interest. The details of the overtaking determination will be described later.

The image processing device 122 performs pre-processing such as logarithmic transformation, sensitivity correction, and the like for measurement data of main scanning that was input from the data acquisition system 107, generates projection data required for image reconstruction, and then reconstructs tomographic images of an object using the projection data in a case where the overtaking determination unit 128 determines that a current scanning position does not overtake contrast medium. The tomographic images reconstructed by the image processing device 122 are stored in the storage device 123, transmitted to the system controller 124, and displayed on the display device 125.

On the other hand, in a case where the overtaking determination unit 128 determines that a current scanning position overtakes contrast medium, the image processing device 122 outputs the determination result to the system controller 124. When obtaining the determination result of that a current scanning position overtakes contrast medium from the overtaking determination unit 128, the system controller 124 resets a scanning condition immediately with the re-scanning control unit 129 and executes re-scanning according to the scanning condition for re-scanning.

A range of the re-scanning includes a position where overtaking occurs (a position where overtaking is determined) to a position where main scanning will terminate.

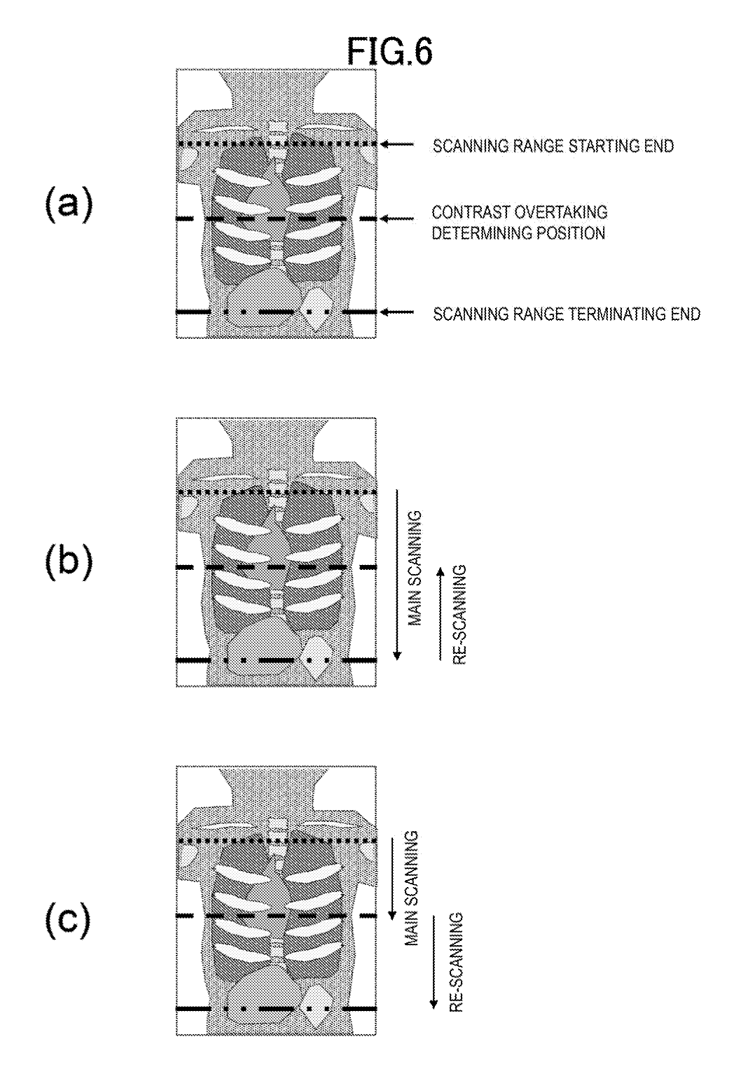

Also, re-scanning may be turnaround scanning in which a bed moves in the reverse direction at a position where main scanning will terminate (turnaround scanning: refer to FIG. 6(b)) or may be started in the same direction as a bed moving direction during main scanning after the overtaking determination unit 128 stops the main scanning at a timing when overtaking is determined and goes back to the position where overtaking was determined and a predetermined waiting time elapses (forward-direction scanning: refer to FIG. 6(c)).

Also, in re-scanning, it is desirable to set a scanning condition such as a bed speed, a rotation speed, a helical pitch, and an X-ray condition so that image quality of re-scanning is equal to that of main scanning before overtaking occurs.

It is desirable that the re-scanning condition (a scanning direction, a waiting time before starting re-scanning, whether or not to perform a monitoring scan, a bed moving speed, a helical pitch, and the like) are specified by an operator in advance before main scanning. A specific example of the re-scanning condition will be described later.

Next, referring to FIGS. 3 to 8, operations of the X-ray CT apparatus 1 will be described.

The system controller 124 of the X-ray CT apparatus executes contrast enhanced scanning processing according to the procedure shown in the flow chart of FIG. 3. That is, the system controller 124 reads out programs and data related to the contrast enhanced scanning processing from the storage device 123 and executes processes based on the programs and the data.

In the contrast enhanced scanning processing, the X-ray CT apparatus 1 first performs positioning scanning (Step S101).

Contrast medium is not used for the positioning scanning because it is desired to perform the positioning scanning at a low dosage from the viewpoint of exposure reduction. The positioning scanning may be scanogram scanning that irradiates X-rays to an object from a given direction and moves the bed 105 in parallel in the body axis direction or may be a helical scan. The helical scan may desirably be a trajectory synchronization helical scan that corresponds to a trajectory of main scanning for overtaking determination to be described later.

The image processing device 122 of the operation console 120 stores measurement data acquired by positioning scanning in a state of raw data in the storage device 123. Also, the image processing device 122 generates a scanogram image using the measurement data acquired by positioning scanning, stores it in the storage device 123, and then displays it on the display device 125 of the operation console 120. The scanogram image is referred to when a scanning condition is set in Step S102, when a scanning range is determined, and the like.

The system controller 124 receives input of a scanning condition and a reconstruction condition (Step S102). As the scanning condition, the system controller 124 receives input of both the scanning conditions for main scanning and for re-scanning in a case where overtaking of contrast medium occurs.

The scanning condition for main scanning includes an X-ray condition such as a tube current and a tube voltage, a scanning range for the main scanning, a helical pitch, a scanning speed, whether or not to require overtaking determination of contrast medium, a threshold value for the overtaking determination of the contrast medium, and the like.

Also, the scanning condition for re-scanning include a waiting time before re-scanning, a scanning direction for re-scanning, an X-ray condition such as a tube current and a tube voltage for re-scanning, a helical pitch, a scanning speed, settings for whether or not to perform trajectory synchronization scanning (that corresponds to trajectories of main scanning), and the like.

Additionally received is input of a scanning condition related to a monitoring scan, such as whether or not to require the monitoring scan and a threshold value in the monitoring scan.

The reconstruction condition includes a reconstruction FOV, a reconstruction filter, a reconstruction slice thickness, and the like. Each of the input conditions is stored in the storage device 123.

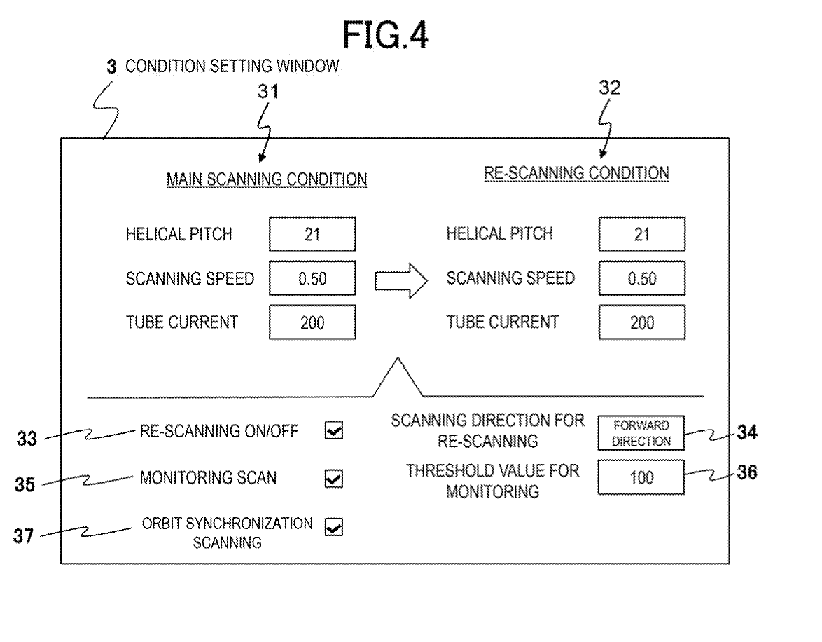

In Step S102, the system controller 124 may display the condition setting window 3 shown in FIG. 4 on the display device 125.

The condition setting window 3 is provided with main scanning condition input fields 31, re-scanning condition input fields 32, a re-scanning ON/OFF setting field 33, a re-scanning direction setting field 34, a monitoring scan setting field 35, a monitoring threshold value setting field 36, a trajectory synchronization scanning setting field 37, and the like. The main scanning condition input fields 31 and the re-scanning condition input fields 32 are respectively provided with the respective input fields in which an operator inputs numerical values of the respective conditions such as a helical pitch, a scanning speed, and a tube current. Additionally, the respective input fields may be provided with a plurality of numerical options.

The re-scanning ON/OFF setting field 33 sets whether or not to perform re-scanning in a case of determining that contrast medium was overtaken in overtaking determination. The re-scanning direction setting field 34 sets whether a bed moving direction in re-scanning is a forward direction or the reverse direction (turnaround scanning) in a case of performing re-scanning. The forward direction is the same as that of main scanning, and the reverse direction is opposite to that of main scanning.

The monitoring scan setting field 35 sets whether or not to perform a monitoring scan of contrast medium. The monitoring threshold value setting field 36 is a setting field to input a threshold value to be used for the monitoring scan. The trajectory synchronization scanning setting field 37 sets whether or not to synchronize trajectories of re-scanning and main scanning with each other.

Hereinafter, described will be a case where overtaking determination is set to “required” in the scanning condition setting process of Step S102.

A scanning condition is set in Step S102 of FIG. 3, contrast medium is injected into an object, the main scanning start is instructed, and then the system controller 124 starts main scanning (contrast enhanced scanning) (Step S103).

The system controller 124 transmits control signals to the X-ray controller 110, the gantry controller 108, and the bed controller 109 based on the scanning condition in Step S103. The X-ray controller 110 controls an electric power to be input to the X-ray source 101 based on the control signals input from the system controller 124. The gantry controller 108 controls a driving system of the rotary disk 102 according to the scanning condition including a rotation speed to rotate the rotary disk 102. The bed controller 109 adjusts a position of the bed 105 to a predetermined scanning start position based on a scanning range and moves the top plate of the bed 105 based on the scanning condition including a bed speed (helical pitch) during scanning.

In main scanning, X-ray irradiation from the X-ray source 101 and measurement of transmission X-ray data by the X-ray detector 106 are repeated together with rotation of the rotary disk 102. The data acquisition system 107 acquires the transmission X-ray data measured by the X-ray detector 106 at various angles (views) around an object (hereinafter, referred to as measurement data) and transmits it to the image processing device 122. The image processing device 122 acquires the measurement data from the data acquisition system 107.

The overtaking determination unit 128 of the image processing device 122 uses the measurement data acquired during main scanning in Step S103 in order to perform overtaking determination of contrast medium (Step S104).

In the overtaking determination of Step S104, there are two methods: (1) Using measurement data acquired by scanogram scanning and (2) Using measurement data of a non-contrast helical scan to be performed before main scanning. In both the cases, difference processing is performed for measurement data acquired by main scanning and measurement data that was acquired by previous non-contrast enhanced scanning and stored in the storage device 123 between data whose scanning positions (body-axis direction positions and viewing angles) are the same, and then overtaking determination is performed based on the differential data.

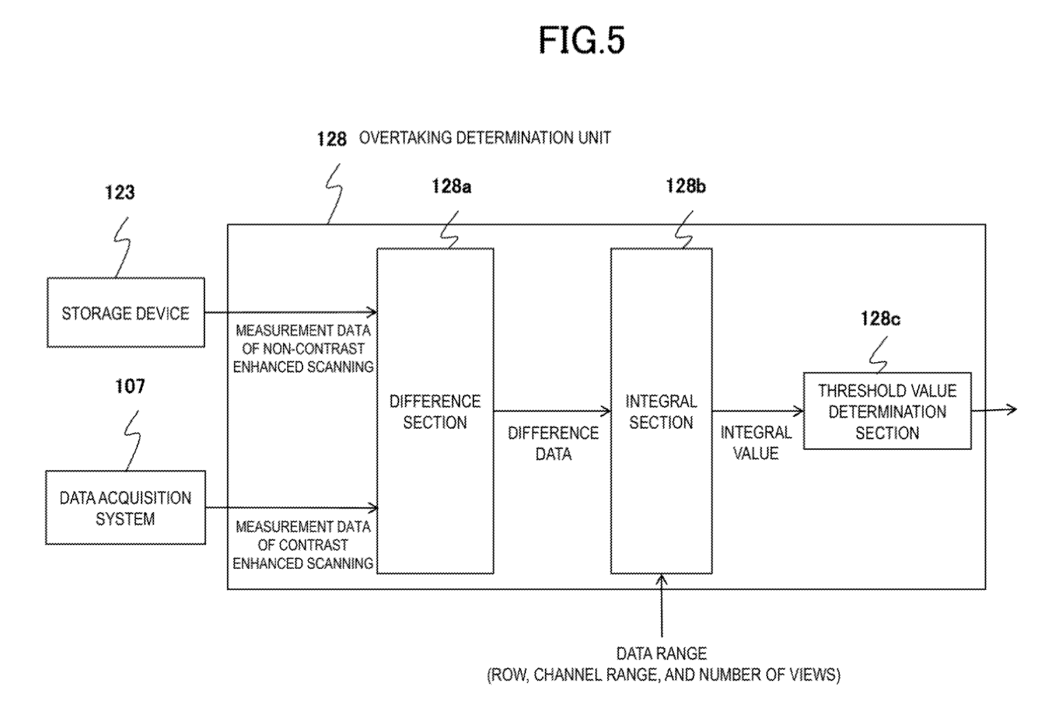

As shown in FIG. 5, the overtaking determination unit 128 has a difference section 128a that performs difference processing for measurement data between non-contrast enhanced scanning and contrast enhanced scanning (main scanning), an integral section 128b that performs integral processing for the differential data obtained by the difference section 128a in a predetermined projection data range, and a threshold value determination section 128c that compares an integral value obtained by the integral section 128b with a predetermined threshold value.

When a helical scan is used for main scanning and overtaking determination is performed using measurement data acquired by scanogram scanning (hereinafter, referred to as scanogram measurement data), the difference section 128a performs difference processing for measurement data whose scanning positions (the body-axis direction position and the view angle) are the same with each other. The measurement data whose scanning positions the body-axis direction position and the view angle) are the same exists once per rotation.

Additionally, in a case where a helical scan is used for main scanning and overtaking determination is performed using scanogram measurement data, the overtaking determination can also be performed using the scanogram measurement data of all the views. In this case, correction processing according to the height of a bed needs to be performed for the scanogram measurement data in advance.

For example, in a case of performing main scanning (a helical scan) in a state where an object is not located in the center of the rotary disk (the measurement center), distances are different between the X-ray source and the object for each view angle, which causes differences also in measurement data to be obtained during one rotation. Therefore, scanogram measurement data for which difference processing is performed is corrected for each view in advance according to a distance between the bed and the tube in the helical scan. The difference section 128a performs difference processing between the measurement data of main scanning and the scanogram measurement data after the correction processing in the same scanning position (the body-axis direction position and the view angle) with each other.

When a helical scan is used for main scanning and overtaking determination is performed using measurement data acquired by a non-contrast trajectory synchronization helical scan, the difference section 128a performs difference processing between measurement data of main scanning and measurement data of the non-contrast trajectory synchronization helical scan whose scanning positions (the body-axis direction position and the view angle) are the same. The overtaking determination can be performed for these measurement data in all the views. The view Intervals for performing the overtaking determination may be set arbitrarily.

Also, in a case where non-contrast measurement data whose trajectory is different from that of main scanning is measured in advance, the non-contrast measurement data with a different trajectory can also be used for the overtaking determination. In this case, tomographic images are reconstructed based on the non-contrast measurement data with a different trajectory, and performing forward projection processing for the tomographic images can virtually generate non-contrast measurement data in which trajectories of main scanning correspond to each other as trajectory synchronization data. The difference section 128a may perform difference processing between trajectory synchronization non-contrast measurement data that was virtually generated in the above procedure and the measurement data of main scanning.

The integral section 128b of the overtaking determination unit 128 performs integral processing for differential data obtained by difference processing in a predetermined data range. It is desirable that the data range for the integral processing is determined according to the numbers of rows, channels, and scanning views of the X-ray detector 106. For example, in a case where the X-ray detector 106 of the X-ray CT apparatus 1 to be used for scanning obtains measurement data having detection elements with 64 rows, 1,000 channels, and 1,000 views per rotation, it should be set so as to integrate differential data of each element of 500 channels in the center, four rows in the center, and one view (the said view). Additionally, the data range for the integral processing is an example, and the values are not limited.

The threshold value determination section 128c determines whether or not a scanning position has overtaken a position of contrast medium by comparing an integral value obtained by the integral section 128b with a predetermined threshold value. When the integral value is larger than the predetermined threshold value, the integral section 128b outputs a determination result showing “not overtaken” because there is the contrast medium in the scanning position. When the integral value is equal to or less than the predetermined threshold value, the integral section 128b outputs a determination result showing “overtaken” to the system controller 124.

Alternatively, the threshold value determination section 128c may determine overtaking by comparing with not an integral value at a scanning position to be a target for the overtaking determination (hereinafter, referred to as said integral value) but an integral value at a scanning position forwarder in the traveling direction (hereinafter, referred to as a forward integral value). The threshold value determination section 128c outputs a determination result showing “overtaken” when a difference between the said integral value and the forward integral value is equal to or more than a predetermined value. When a difference between the said integral value and the forward integral value is within the predetermined value, the threshold value determination section 128c outputs a determination result showing “not overtaken” to the system controller 124.

The description goes back to FIG. 3.

When a determination result showing “overtaking” is obtained in Step S104 (Step S104: OVERTAKEN), the system controller 124 sets a scanning condition to perform re-scanning for a range from a position where overtaking was determined to a scanning range terminating end (Step S105). In the re-scanning, the scanning condition is set in consideration with arrival of contrast medium.

Re-scanning may be turnaround scanning as shown in FIG. 6(b) or may be forward-direction scanning as shown in FIG. 6(c).

When turnaround scanning is used as re-scanning, the system controller 124 continues scanning until a scanning range terminating end 43 set as a main scanning range as shown in FIG. 6(b), waits for arrival of contrast medium after the scanning until the scanning range terminating end 43 ends, and then sets a scanning condition to perform the re-scanning by turning around in the reverse direction.

Also, when turnaround scanning is used as re-scanning, the system controller 124 continues scanning until the scanning range terminating end set as a main scanning range as shown in FIG. 6(b), performs a monitoring scan at the scanning range terminating end 43 after the scanning until the scanning range terminating end ends, and may start re-scanning at a time point when a CT value of a monitoring region is equal to or more than a predetermined threshold value. In this case, it is desirable that the re-scanning (turnaround scanning) is performed at a higher speed in order to prevent unevenness of contrast medium.

Also, it is desirable that there is no difference from an image noise amount of scanning in which overtaking was caused. Therefore, in re-scanning, while scanning is being performed for a re-scanning range in a shorter time by increasing one or more of a bed moving speed and a scanning speed, one or more of a tube current, a bed moving speed, and a scanning speed are adjusted. Hence, it is desirable that a scanning condition is reset so that image noises are equal between main scanning and re-scanning.

Also, when turnaround scanning is used as re-scanning, it is desirable that the trajectory of the turnaround scanning is synchronized with that of main scanning.

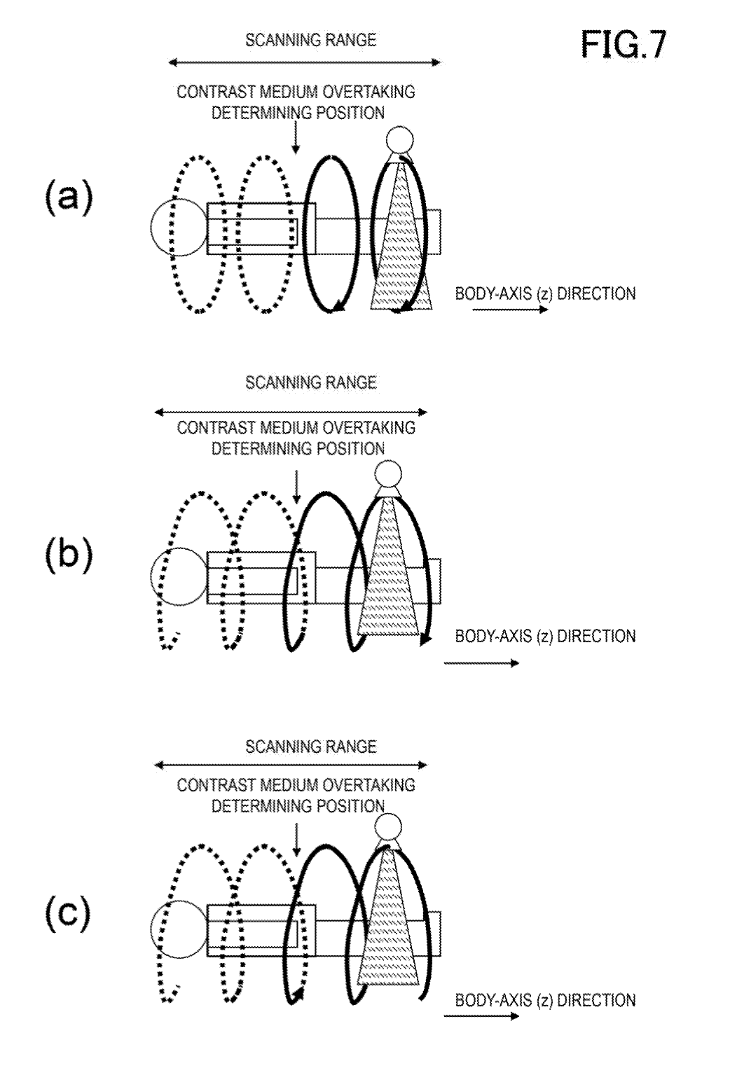

FIG. 7 is a diagram explaining trajectories in re-scanning.

As shown in FIG. 7(a), when an axial scan is used for main scanning, the axial scan may be performed so as to irradiate an X-ray in the same body-axis direction position as the main scanning also in turnaround scanning.

When a helical scan is performed in main scanning as shown in FIG. 7(b), turnaround scanning performs scanning in which the trajectory is synchronized while the bed moving direction is set in the reverse direction as shown in FIG. 7(c). That is, scanning is performed by reversely rotating the rotary disk 102 to the main scanning. However, in a case where the rotation direction of the rotary disk. 102 is normally limited to one direction due to the structure, the turnaround scanning is performed without performing trajectory synchronization scanning.

Also, when forward-direction scanning is used as re-scanning, the system controller 124 interrupts main scanning at a time point when a determination result showing “overtaken” is obtained as shown in FIG. 6(c), returns to the overtaking determination position to wait for a predetermined waiting time, and then sets a scanning condition to start re-scanning in the forward direction. It is desirable that a trajectory of the re-scanning is synchronized with that of the main scanning. Also, by reducing a helical pitch and a scanner rotation speed or the like, the scanning condition may be reset so that overtaking does not occur again.

A waiting time until re-scanning starts (a time to wait for arrival of contrast medium) may be set as, for example, a waiting time set in advance by an operator when a scanning condition of Step S102 is set, may be set as a time until a difference between a monitoring image acquired by a monitoring scan in a predetermined position and an image previously reconstructed by non-contrast measurement data is equal to or more than a predetermined threshold value, or may be determined in consideration with a waiting time until a contrast effect reaches the peak based on a CT value change in the monitoring image acquired by the monitoring scan and a CT value of a contrast section.

There is a case where image quality discontinuity occurs due to a difference between hours of scanning in the body-axis direction in a boundary position between main scanning and re-scanning. Therefore, the image quality discontinuity may be reduced by partially superimposing scanning ranges of the main scanning and the re-scanning to perform weighted addition for the superimposed range of both the scanning data.

The weighted addition may be performed between measurement data of main scanning and re-scanning or between image data of reconstruction processes. In a case of performing the weighted addition between the measurement data, the reconstruction processes are performed for the measurement data for which the weighted addition was performed.

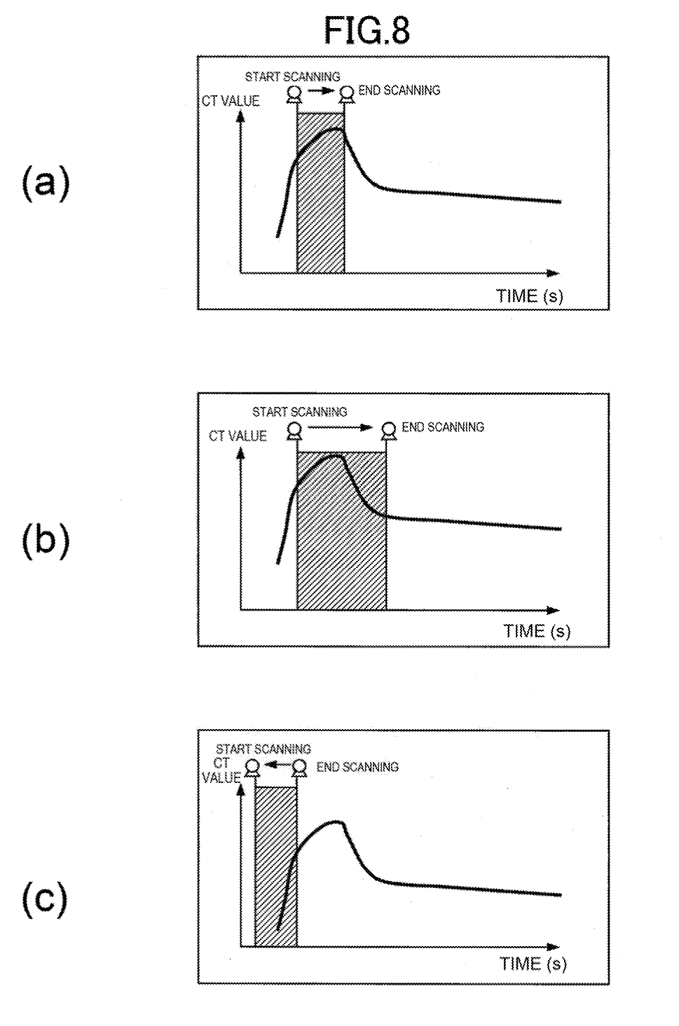

Referring to FIG. 8, a relationship between a flowing speed of contrast medium, a bed moving speed, and a contrast effect will be described.

FIG. 8 is an example of Time Density Curve (hereinafter, referred to as TDC) during contrast enhanced scanning in an aortic position.

Curves (TDCs) shown in FIG. 8 show relationships between time and a CT value (contrast effect) in a case where there is no bed movement. When contrast medium arrives at a scanning position, the CT value increases drastically and decreases drastically after the peak. As shown in FIG. 8(a), it is desirable that scanning is performed at a timing where a contrast effect is as high as possible during contrast enhanced scanning. However, in a case where the bed 105 moves in the same direction as the flow of the contrast medium, when a bed moving speed is faster than a flowing speed of the contrast medium, scanning is performed in a position where the contrast effect exceeded the peak as shown in FIG. 8(c), which cannot obtain a desired contrast result. Inversely, when the bed moving speed is too slow, scanning at a timing after the contrast peak is included as shown in FIG. 8(b), which also cannot obtain a desired contrast result.

Therefore, it is desirable that a bed moving speed during main scanning is set so as not to be excessively slower than a flowing speed of contrast medium. Also, as shown in FIG. 8(c), when the bed moving speed is considerably faster than the flowing speed of the contrast medium, it is desirable to delay a timing of starting the main scanning according to the required time for scanning. Specifically, it is desirable to set a later time in a range where a desired contrast effect can be obtained as a timing of starting the scanning.

Additionally, overtaking determination may be predictively performed based on a relationship between a bed moving speed and a contrast effect peak as shown in FIG. 8.

That is, the overtaking determination unit 128 determines whether a scanning position is getting closer to, getting away from, or keeping the contrast effect peak based on the temporal change of differential data in order to predict overtaking. The scanning condition setting unit 126 may adjust a scanning condition based on the overtaking prediction result. For example, in a case where the overtaking determination unit 128 determines that a current scanning position is gradually getting closer to a peak position of the contrast effect based on the differential data change, a bed speed is delayed because contrast medium will be overtaken at such a bed speed. Inversely, in a case where the scanning position is getting away from the peak, the bed speed is increased. Thus, the scanning condition may be adjusted by predictively determining overtaking before it actually occurs.

Also, in a case of performing forward-direction scanning shown in FIG. 6(c) during re-scanning, there is a case where overtaking contrast medium occurs again when the re-scanning is performed at the same bed moving speed and the same scanning speed as main scanning. Therefore, it is desirable to perform overtaking determination of Step S104 also while the re-scanning is being executed (Step S105->Step S104). In a case where overtaking the contrast medium occurs during the re-scanning, the system controller 124 brings a bed position back to the position where overtaking occurred (where overtaking was determined), resets a scanning condition, and then performs re-scanning after a predetermined waiting time elapsed. Thus, it may be configured so as to repeatedly perform re-scanning in the forward direction by always taking arrival of the contrast medium into account.

Also, in re-scanning, the scanning speed may be reduced while the trajectories are being synchronized by delaying a bed moving speed, a scanning speed, or the like. In this case, overtaking contrast medium again can be prevented. In a case of delaying a bed moving speed and a scanning speed, it is desirable to take an influence on image quality by reducing an X-ray tube current.

When main scanning and re-scanning for a scanning range end, the image processing device 122 performs an image reconstruction process based on the acquired transmission X-ray data (Step S106).

When a trajectory of re-scanning is corresponding (synchronized) with that of main scanning, measurement data obtained by the main scanning is converted with that obtained by the re-scanning and can be used for image reconstruction as a series of data.

On the other hand, when the trajectory of re-scanning is not corresponding (synchronized) with that of main scanning, the image processing device 122 performs image reconstruction processes respectively based on each measurement data obtained by the main scanning and the re-scanning. Final tomographic images can be generated by combining images generated from the measurement data of the main scanning with that generated from the measurement data of the re-scanning.

The system controller 124 stores reconstructed images in the storage device 123, displays them on the display device 125, and then ends a series of contrast enhanced scanning processes.

As described above, the X-ray CT apparatus 1 of the present invention determines that a scanning position overtakes contrast medium position during contrast enhanced scanning. When overtaking is determined, a scanning condition is reset in consideration with arrival of the overtaking contrast medium, and then re-scanning is executed under the reset scanning condition. Therefore, in a case where overtaking of the contrast medium occurs, the contrast examination can be continued by resetting an appropriate scanning condition using the overtaking contrast medium as is. Hence, repeated contrast examinations can be avoided, which can reduce the burden on an object.

Also, the overtaking determination of contrast medium is performed based on differential data between measurement data obtained by non-contrast enhanced scanning to be performed before main scanning and that obtained by the main scanning. Therefore, images do not need to be reconstructed during scanning to check whether or not there is a contrast effect, which can perform overtaking determination at a high speed. Hence, overtaking of the contrast medium can be detected at an early timing, which can swiftly shift to re-scanning while the contrast medium is within a scanning range. Also, a scanning condition for the re-scanning are adjusted so as to be the same level as image quality of the main scanning, and this can reconstruct images whose image quality is even in the entire scanning range.

Although a suitable embodiment of the X-ray CT apparatus 1 related to the present invention has been described above, the present invention is not limited to the above embodiment. It is apparent that a person skilled in the art could arrive at various modified examples or amended examples within the scope of the technical ideas disclosed in the present application, and it is understood that these naturally belong to the technical scope of the present invention.

### REFERENCE SIGNS LIST

1: X-ray CT apparatus
100: scan gantry unit
101: X-ray source
102: rotary disk
106: X-ray detector
120: operation console
121: input device
122: image processing device
123: storage device
124: system controller
125: display device
126: scanning condition setting unit
127: scanning control unit
128: overtaking determination unit
129: re-scanning control unit

1. An X-ray CT apparatus comprising:
an X-ray source that irradiates X-rays to an object;
an X-ray detector that is disposed opposite to the X-ray source and detects the X-rays transmitted through the object;
a rotary disk that is provided with the X-ray source and the X-ray detector and rotates around the object;
an image reconstruction unit that reconstructs an image based on the transmitted X-ray data detected by the X-ray detector;
a scanning condition setting unit that sets a scanning condition of main scanning to be performed by injecting contrast medium into the object;
a scanning control unit that executes the main scanning under the scanning condition set by the scanning condition setting unit;
an overtaking determination unit that determines whether or not a scanning position overtakes a position of the contrast medium during executing the main scanning; and
a re-scanning control unit that resets the scanning condition and executes re-scanning under the reset scanning condition in a case where overtaking is determined by the overtaking determination unit.

2. The X-ray CT apparatus according to claim 1,
wherein the overtaking determination unit determines the overtaking based on differential data between measurement data acquired by non-contrast enhanced scanning to be performed before the main scanning and measurement data to be acquired by the main scanning.

3. The X-ray CT apparatus according to claim 2,
wherein the non-contrast enhanced scanning is positioning scanning, and the overtaking determination unit acquires the differential data by performing difference processing, in which the target is measurement data to be acquired by the main scanning and that for the positioning scanning, between the measurement data whose body-axis direction position and view position are the same.

4. The X-ray CT apparatus according to claim 2,
wherein the non-contrast enhanced scanning is trajectory synchronization scanning in which trajectories of the main scanning correspond to each other, and
the overtaking determination unit acquires the differential data by performing difference processing, in which the target is measurement data to be acquired by the main scanning and that for the non-contrast enhanced scanning, between the measurement data whose body-axis direction position and view position are the same.

5. The X-ray CT apparatus according to claim 1,
wherein a range of the re-scanning includes a position where the overtaking occurs to a position where the main scanning will terminate.

6. The X-ray CT apparatus according to claim 1,
wherein the re-scanning control unit synchronizes a trajectory of the re-scanning with that of the main scanning.

7. The X-ray CT apparatus according to claim 1,
wherein the re-scanning control unit sets a scanning condition for the re-scanning to perform turnaround scanning in which a bed moving direction is changed to the reverse direction at a position where the main scanning will terminate.

8. The X-ray CT apparatus according to claim 1,
wherein the scanning control unit stops the main scanning at a timing when the overtaking determination unit determines overtaking, and
the re-scanning control unit sets a scanning condition for the re-scanning to start re-scanning in the same direction as a bed moving direction during the main scanning after a predetermined waiting time elapses from the timing when the overtaking determination unit determined overtaking.

9. The X-ray CT apparatus according to claim 1,
wherein the re-scanning control unit sets a scanning condition for the re-scanning so that image quality between the main scanning before overtaking occurs and the re-scanning is equivalent.

10. A contrast enhanced scanning method of an X-ray CT apparatus including:
executing main scanning that is performed by injecting contrast medium into an object under set a scanning condition;
determining whether or not a current scanning position overtakes a position of contrast medium during the main scanning;
resetting the scanning condition in a case where overtaking is determined; and
executing re-scanning under the reset scanning condition.
